(19) United States
(12) Patent Application Publication
(10) Pub. No.: US 2026/0321402 A1
(43) Pub. Date: Oct. 15, 2026

(54) SYSTEM AND METHOD FOR MANAGING VARIANCE EVENTS IN HEALTHCARE

(71) Applicant: EXPANDED EXISTENCE, INC., Ocoee, FL (US)

(72) Inventors: ROBERT L. MASSON, WINDERMERE, FL (US); Nicholas CAMBATA, Ashburn, VA (US)

(21) Appl. No.: 19/174,625

(22) Filed: Apr. 9, 2025

(51) Int. Cl. G16H 40/20; G06F 40/40; G16H 70/20

(52) U.S. Cl. CPC G16H 40/20; G16H 70/20; G06F 40/40

(57) ABSTRACT

A system for managing variance events in healthcare is described. The system includes a user device, and a server communicatively coupled to the user device. The user device is configured to receive data indicating an occurrence of a variance event in a medical room of a medical facility and formulate a query based on the received data. The server is configured to access a large language model (LLM) associated with the healthcare and fine-tune the LLM based on a plurality of historical datasets to provide a long-term context to the LLM. The server is further configured to provide the query to the fine-tuned LLM, generate a response associated with the query from the LLM, and control one or more medical devices in the medical room to manage the variance event based on the response. The response indicates one or more actions to be performed to manage the variance event.

### BACKGROUND OF THE INVENTION

According to data from the United States Surgical Procedures Market Report 2022, over one hundred (100) million surgical procedures were performed in 2022 in the United States alone, and that number continues to grow. In medical healthcare, many emergency events may occur unexpectedly. The approach to handling such emergencies varies depending on various factors, such as location, availability of medical practitioners, and the facilities provided. If an emergency event occurs and the medical practitioner is unavailable, or the available staff lacks experience, the medical practitioner or staff refers to standard emergency protocols or standard surgical checklists. While these resources provide guidance, they are not always effective in managing complex or high-risk cases, potentially leading to adverse outcomes, including life-threatening complications.

### BRIEF DESCRIPTION OF THE SEVERAL VIEWS OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention and explain various principles and advantages of those embodiments.

FIG. 1 illustrates a system for managing variance events in healthcare, in accordance with some embodiments;

FIG. 2 illustrates a block diagram of an exemplary user device for use within the system of FIG. 1, in accordance with some embodiments;

FIG. 3 illustrates a block diagram of an exemplary server for use within the system of FIG. 1, in accordance with some embodiments;

FIG. 4 illustrates a process map for managing the variance events in healthcare, in accordance with some embodiments; and

FIG. 5 illustrates an exemplary method for managing the variance events in healthcare, in accordance with some embodiments.

Skilled artisans will appreciate that elements in the figures are illustrated for simplicity and clarity and have not necessarily been drawn to scale. For example, the dimensions of some of the elements in the figures can be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the description with details that will be readily apparent to those of ordinary skill in the art having the benefit of the description herein.

### DETAILED DESCRIPTION OF THE INVENTION

In one aspect, a system for managing variance events in healthcare is described. The system includes a user device, and a server communicatively coupled to the user device. The user device is configured to receive data indicating an occurrence of a variance event in a medical room of a medical facility and formulate a query indicating the occurrence of the variance event in the medical room of the medical facility based on the received data. The server is configured to access a large language model (LLM) associated with the healthcare and fine-tune the LLM based on a plurality of historical datasets to provide a long-term context to the LLM. The historical dataset includes one or more of a plurality of surgical profiles identifying one or more steps of a plurality of medical procedures. Each surgical profile of the plurality of surgical profile is associated with a medical practitioner identifier identifying a medical practitioner, a medical procedure identifier identifying a medical procedure, a facility identifier identifying a medical facility, and a medical room identifier identifying a medical room in a corresponding medical facility. The historical dataset further includes a facility knowledge dataset associated with a plurality of medical facilities. The facility knowledge dataset includes data associated with a layout and a setup of one or more rooms in each medical facility of the plurality of medical facilities. The historical dataset further includes a surgeon knowledge dataset associated with a plurality of medical practitioners and insights from a plurality of historical medical procedures performed by each medical practitioner of the plurality of medical practitioners. The surgeon knowledge dataset is classified based on genealogy of the plurality of medical practitioners. The server is further configured to provide the query to the fine-tuned LLM, generate a response associated with the query from the LLM, and control one or more medical devices in the medical room to manage the variance event based on the response. The response indicates one or more actions to be performed to manage the variance event.

In another aspect, a method for managing variance events in healthcare is described. The method includes receiving, by a user device, a query indicating an occurrence of a variance event in a medical room of a medical facility and formulating, by the user device, a query indicating the occurrence of the variance event in the medical room of the medical facility based on the received data. The method further includes accessing, by a server, a large language model (LLM) associated with the healthcare and fine-tuning, by the server, the LLM based on a plurality of historical datasets to provide a long-term context to the LLM. The historical dataset includes one or more of a plurality of surgical profiles identifying one or more steps of a plurality of medical procedures. Each surgical profile of the plurality of surgical profile is associated with a medical practitioner identifier identifying a medical practitioner, a medical procedure identifier identifying a medical procedure, a facility identifier identifying a medical facility, and a medical room identifier identifying a medical room in a corresponding medical facility. The historical dataset further includes a facility knowledge dataset associated with a plurality of medical facilities. The facility knowledge dataset includes data associated with a layout and a setup of one or more rooms in each medical facility of the plurality of medical facilities. The historical dataset further includes a surgeon knowledge dataset associated with a plurality of medical practitioners and insights from a plurality of historical medical procedures performed by each medical practitioner of the plurality of medical practitioners. The surgeon knowledge dataset is classified based on genealogy of the plurality of medical practitioners. Further, the method includes providing, by the server, the query to the fine-tuned LLM, generating, by the server, a response associated with the query from the LLM, and controlling, by the server, one or more medical devices in the medical room to manage the variance event based on the response. The response indicates one or more actions to be performed to manage the variance event.

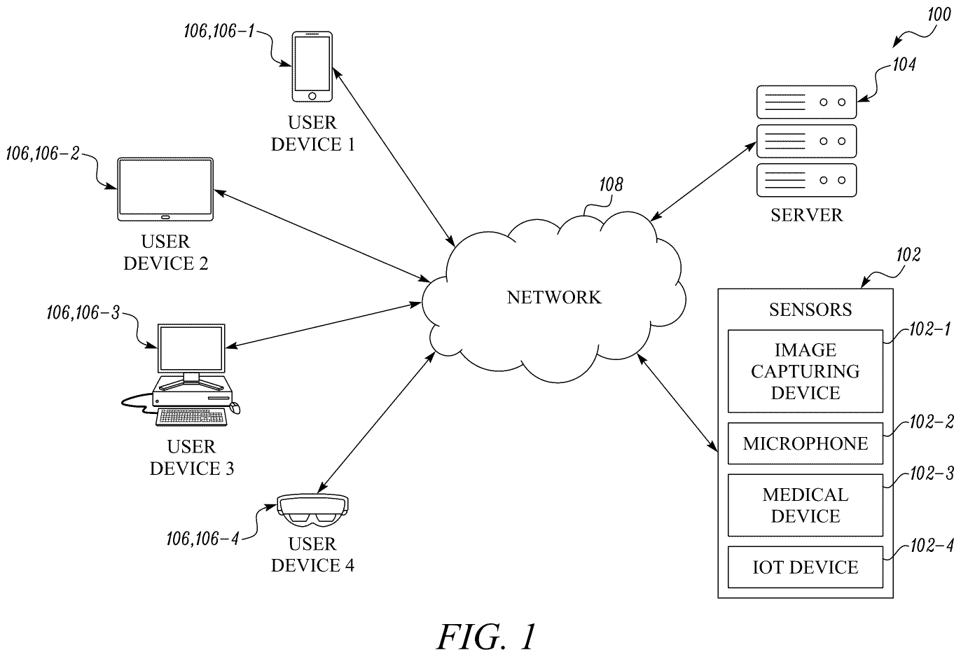

Referring to FIG. 1, an exemplary system 100 for managing variance events in healthcare is illustrated. The healthcare corresponds to a system for providing and receiving medical treatment, for example, by diagnosing, treating, preventing, or managing diseases and medical conditions of individuals. The healthcare includes various medical procedures involving a series of steps carried out on an individual to improve health, treat a disease or injury, manage medical conditions, or make a diagnosis. For example, the medical procedure includes, but is not limited to, spinal surgery, scoliosis reduction, heart surgery, orthopedics, biopsy, or any type of procedure to diagnose, treat, or manage a medical condition, now known or in the future developed. The healthcare also includes set-up of a medical room corresponding to an arrangement including, but not limited to, a location, a position, an orientation, and an order of placement of medical items in the medical room.

The variance event in the healthcare refers to any deviation, discrepancy, or unexpected occurrence from the standard, planned, or expected course of medical care, treatment, or procedure. For example, the variance event includes deviations from established protocols prior, during, or after the execution of medical procedures, such as surgical interventions, medication administration, or diagnostic tests. In some embodiments, the variance event includes discrepancies in usage, set-up, and arrangement of medical devices, supplies, and procedure rooms. Variance events occurs when actual procedures deviate from established protocols. Additionally, variance events also include patient outcomes, vital signs, or medical conditions that fall outside the expected range. In accordance with various embodiments, the system 100 is configured to manage such variance events in the healthcare by receiving a query indicating an occurrence of the variance event in a medical room of a medical facility and generating a response indicating one or more actions to be performed to manage the variance event. The medical facility refers to any establishment or institution that provides healthcare services, including hospitals, clinics, nursing homes, rehabilitation centers, laboratories, and pharmacies. The medical rooms in the medical facility refer to designated areas or spaces for patient care, treatment, and procedures, such as examination rooms, operating rooms, recovery rooms, and consultation rooms.

The system 100 includes one or more sensors 102 (for example, but not limited to, the sensors 102-1, 102-2, 102-3, and 102-4), a server 104, and at least one user device 106 (for example, but not limited to, the user devices 106-1, 106-2, 106-3, and 106-4). The sensors 102, the server 104, and the user devices 106 are communicatively coupled to each other via a communication network 108 (referred also interchangeably as network 108). The network 108 is a secure network including, but not limited to, a Local Area Network (LAN), a Wireless Local Area Network (WLAN), a Wireless Personal Area Network (WPAN), a Small Area Network (SAN), a telecommunication network including, but not limited to, a fourth generation (4G) and a fifth generation (5G) cellular network, and other wireless communication networks employing radio frequency (RF) signals, or other forms of wireless communication, and utilizing any of a variety of communications protocols, as is now known or in the future developed.

In accordance with various embodiments, each sensor 102 is configured to capture data associated with the occurrence of the variance event in the medical room of the medical facility. For example, the sensors 102 are installed in the medical room of the medical facility. The sensor 102 includes, but is not limited to, one or more of an image capturing device 102-1, a microphone 102-2, a medical device 102-3, an internet of things (IoT) device 102-4, or any other sensing unit capable of capturing the data associated with the variance event. In an exemplary embodiment, the image capturing device 102-1 is configured to capture the data, for example, visual data associated with the occurrence of the variance event in the medical room of the medical facility. The visual data corresponds to images or video frames of the medical room capturing any deviation, discrepancy, or unexpected occurrence from the standard, planned, or expected course of medical care, set-up, treatment, or procedure. In some embodiments, the visual data corresponds to a visual indication provided on a screen in the medical room. The image capturing device 102-1 includes cameras, such as monochrome digital cameras, high-resolution digital cameras, or any suitable digital cameras. Further, the cameras include still cameras, camcorders, video cameras or Closed-Circuit Television (CCTV) cameras, without any limitations. The cameras also include optical flow chips that facilitate acquisition of the visual data. In some examples, the camera embodies a Complimentary Metal-Oxide Semiconductor (CMOS) cameras. Although not shown, in some embodiments, the image capturing devices 102-1 include 3-dimensional (3D) sensors, Light Detection and Ranging (LIDAR) sensors, and other devices known to persons skilled in the art, to capture the visual data.

In an exemplary embodiment, the microphone 102-2 is configured to capture the data, for example, audio data associated with the occurrence of the variance event in the medical room of the medical facility. For example, the audio data corresponds to an emergency alarm, a siren, or a conversation between staff members in the medical room. In an exemplary embodiment, the medical device 102-3 is configured to capture data, for example, any deviation, discrepancy, or unexpected changes in patient outcomes, vital signs, or medical conditions that fall outside an expected range. For example, the data indicates that the blood pressure of the patient is outside the expected blood pressure range. The medical device 102-3 includes any medical device used in the healthcare sector, for example but not limited to, anesthesia systems, electrocautery systems, C-arm systems, enabling technology systems or workstations (for example, but not limited to, a microscope, an electrocardiogram device, a robotic surgical system, a navigation system, a networked robotics, an illumination system, or any other enabling technology systems now known or in the future developed, and accompanying monitors or displays), a blood pressure monitoring machine, a heart rate monitoring machine, and various other medical devices (not illustrated) now known or in the future developed. In an exemplary embodiment, the IoT device 102-4 is configured to capture data, for example, any deviation, discrepancy, or unexpected occurrence from the standard, planned, or expected course of medical care, set-up, treatment, or procedure. For example, the IoT device 102-4 is configured to capture deviations in the patient's vital signs, environmental conditions, medication adherence, medical care adherence, and set-up from the standard protocols. For example, the IoT device 102-4 indicates that the medical device, such as, the heart rate monitoring machine has stopped working.

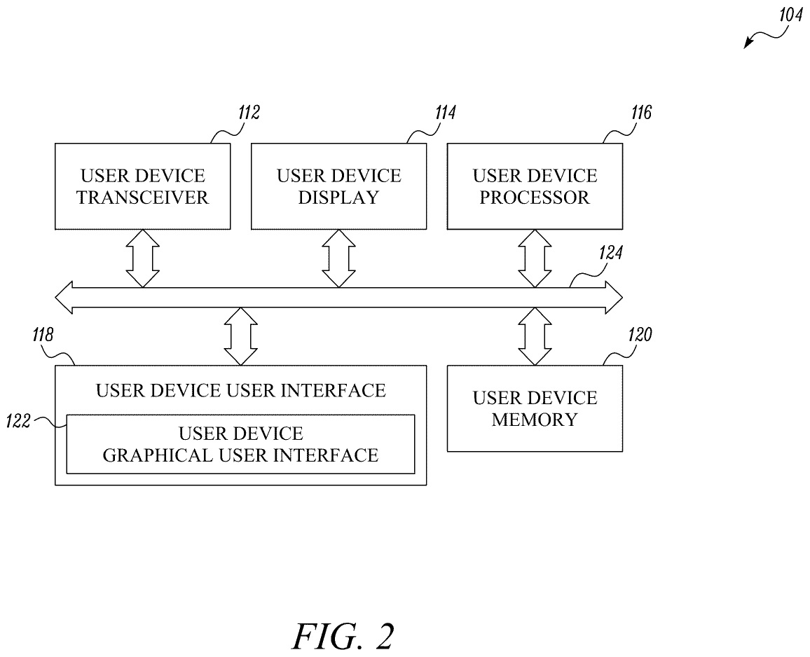

In accordance with various embodiments, the user device 106 operates as an interface for its user to receive data associated with the occurrence of the variance event from the one or more sensors 102 and formulate the query indicating the occurrence of the variance event in the medical room of the medical facility. In some embodiments, the user device 106 is configured to receive the query or supplement data associated with the query from its user. The user corresponds to a nurse, a medical practitioner, a medical intern, or any support staff. The user device 106 corresponds to at least one of a wearable device, a portable device, or any other communication device now known or in the future developed. For example, as illustrated in FIG. 1, the user device 106 is a smartphone 106-1, a tablet 106-2, a computing device 106-3, an augmented reality device 106-4 or any other communication device capable of communicating with the server 104, now known or in the future developed. The various components of user device 106 will now be described hereinafter with respect to FIG. 2. It should be appreciated by those of ordinary skill in the art that FIG. 2 depicts the user device 106 in a simplified manner and a practical embodiment includes additional components and suitably configured logic to support known or conventional operating features that are not described in detail herein. Although the user device 106 is illustrated and described to be implemented within a single communication device, it is contemplated that the one or more components of the user device 106 are alternatively be implemented in a distributed computing environment.

Referring to FIG. 2, the user device 106 includes, among other components, a user device transceiver 112, a user device display 114, a user device processor 116, a user device user interface 118, and a user device memory 120. The components of the user device 106, including the user device transceiver 112, the user device display 114, the user device processor 116, the user device user interface 118, and the user device memory 120, cooperate with one another to enable operations of the user device 106. The components of the user device 106 (for example 112, 114, 116, 118, 120) are communicatively coupled via a user device local interface 124. The user device local interface 124 includes, for example, but is not limited to, one or more buses or other wired or wireless connections, as is now known in the art or in the future developed. In an embodiment, the user device local interface 124 has additional elements, which are omitted for simplicity, such as controllers, buffers (caches), drivers, repeaters, and receivers, among many others, to enable communications. Further, in some embodiments, the user device local interface 124 includes address, control, and/or data connections to enable appropriate communications among the aforementioned components. Although, not shown, in some embodiments, the one or more sensors 102 are integrated within the user device 106.

As illustrated, the user device 106 includes the user device transceiver 112 to transmit one or more inputs to and receive one or more outputs from one or more other devices, (as illustrated in FIG. 1) such as, the sensors 102, the server 104. For example, the user device transceiver 112 is configured to receive the data associated with the occurrence of the variance event in the medical room of the medical facility from the sensors 102 and transmit the query indicating the occurrence of the variance event to and/or receive the response associated with the query from the server 104. In some embodiments, the user device transceiver 112 is configured to transmit feedback associated with the accuracy of the response to the server 104. The user device transceiver 112 includes a transmitter circuitry and a receiver circuitry to enable the user device 106 to communicate with the one or more other devices. In this regard, the transmitter circuitry includes appropriate circuitry to transmit the one or more inputs to the one or more other devices and the receiver circuitry includes appropriate circuitry to receive the one or more outputs from the one or more other devices. It will be appreciated by those of ordinary skill in the art that the user device 106 includes a single user device transceiver as illustrated, or alternatively separate transmitting and receiving components, for example but not limited to, a transmitter, a transmitting antenna, a receiver, and a receiving antenna.

The user device user interface 118 is configured to receive the inputs from and/or provide the outputs to the user. The inputs are provided via a touch screen display (such as, the user device display 114), an image capturing device (such as, a camera), a touch pad, a keyboard, a microphone, a recorder, a mouse, or any other user input mechanism integrated within or coupled to the user device 106, now known or developed in the future. The outputs are provided via a display device (such as the user device display 114), a speaker, a haptic output, or any other output mechanism integrated within or coupled to the user device 106, now known or developed in the future. The user device user interface 118 further includes a serial port, a parallel port, an infrared (IR) interface, a universal serial bus (USB) interface and/or any other interface herein known or developed in the future.

In accordance with some embodiments, the user device user interface 118 includes a user device graphical user interface (GUI) 122 through which the user communicates with the server 104. The user device GUI 122 is the application or the web portal or any other suitable interface for accessing the server 104. The user device GUI 122 includes one or more of graphical elements including, but not limited to one or more of dialogue boxes, window, web forms, text input field, microphone button, camera button, file upload button, text output display window, audio player, image/video display window, and/or the like.

The user device display 114 is configured to display text, images, videos, numbers, infographics, charts, diagrams, motion graphics, typography, dialogue boxes, window, web forms, text input field, microphone button, camera button, file upload button, text output display window, audio player, image/video display window, and other graphical elements now known or developed in future. The user device display 114 includes a display screen, a head-mounted display, or a computer monitor now known or in the future developed. In accordance with some embodiments, the user device display 114 is configured to display on the user device GUI 122 the outputs received from the one or more other devices.

The user device memory 120 is a non-transitory memory configured to store a set of instructions that are executable by the user device processor 116 to perform predetermined operations. For example, the user device memory 120 includes any of the volatile memory elements (for example, random access memory (RAM)), non-volatile memory elements (for example, read only memory (ROM)), and combinations thereof. Moreover, the user device memory 120 incorporates electronic, magnetic, optical, and/or other types of storage media. In accordance with some embodiments, the user device memory 120 is also configured to store the application associated with the user device GUI 122.

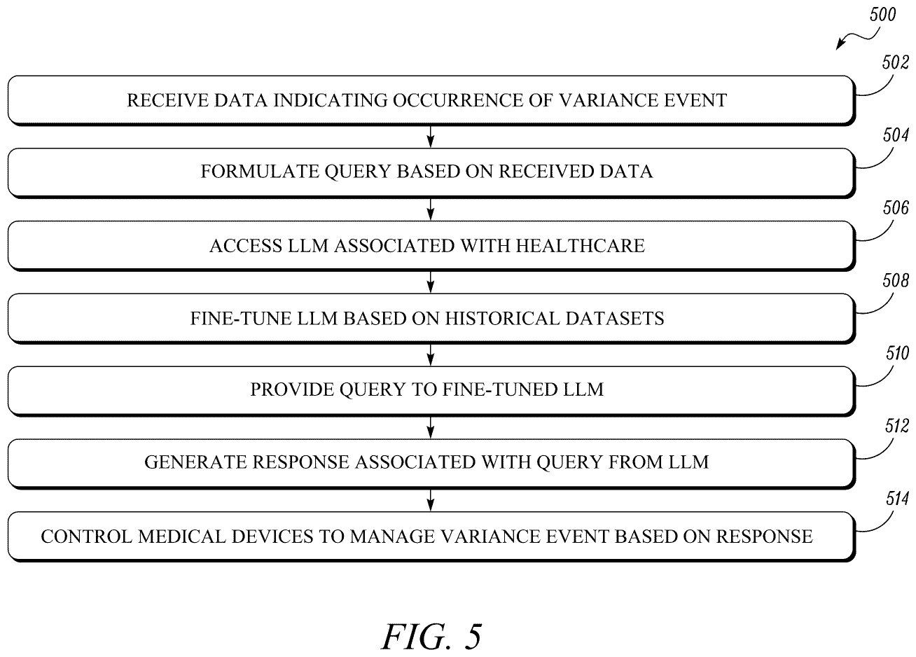

The user device processor 116 is configured to execute the instructions stored in the user device memory 120 to perform the predetermined operations. The user device processor 116 includes one or more microprocessors, microcontrollers, DSPs (digital signal processors), state machines, logic circuitry, or any other device or devices that process information or signals based on operational or programming instructions. The user device processor 116 is implemented using one or more controller technologies, such as Application Specific Integrated Circuit (ASIC), Reduced Instruction Set Computing (RISC) technology, Complex Instruction Set Computing (CISC) technology, or any other similar technology now known or in the future developed. The user device processor 116 is configured to cooperate with other components of the user device 106 to perform operations, as described in detail with reference to FIG. 5.

In accordance with various embodiments, the user device processor 116 is configured to obtain the data associated with the occurrence of the variance event in the medical room of the medical facility and formulate the query indicating the occurrence of the variance event based on the obtained data. For example, the query includes the data associated with the occurrence of the variance event in the medical room of the medical facility captured by the sensors 102. In such cases, the server 104 is configured to process the query and identify the variance event (for example, any deviation, discrepancy, or unexpected occurrence from the standard, planned, or expected course of medical care, set-up, treatment, or procedure) based on a comparison of the captured data with the standard, planned, or expected course of medical care, set-up, treatment, or procedure. In some embodiments, the user device processor 116 is configured to process the data received from the sensors 102 and determine any deviation, discrepancy, or unexpected occurrence from the standard, planned, or expected course of medical care, set-up, treatment, or procedure based on a comparison of the captured data with the standard, planned, or expected course of medical care, set-up, treatment, or procedure. The user device processor 116 is further configured to formulate the query based on the determined deviation, discrepancy, or unexpected occurrence. In some embodiments, the user device processor 116 is configured to receive the query and/or the supplement data associated with the query from its user. For example, the user device processor 116 is configured to receive the data indicating the drop in the blood pressure of a patient from the sensor 102 along with the supplement data from its user providing information regarding deviations in other vital signs of the patient.

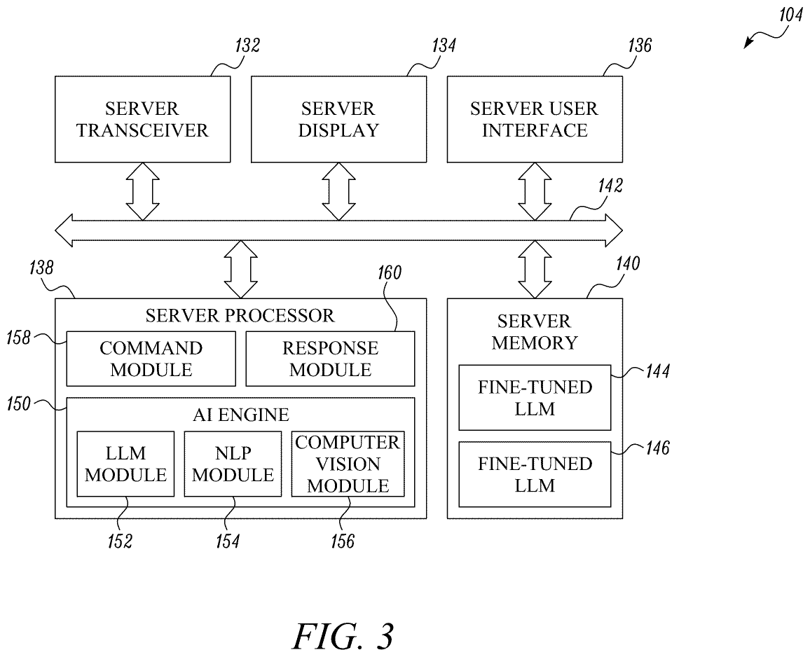

Referring back to FIG. 1, the server 104 is configured to generate the response indicating one or more actions to be performed to manage the variance event. In some embodiments, the server 104 is also configured to control one or more medical devices in the medical room to manage the variance event based on the response. The various components of the server 104 will now be described hereinafter with respect to FIG. 3. It should be appreciated by those of ordinary skill in the art that FIG. 3 depicts the server 104 in a simplified manner and a practical embodiment includes additional components and suitably configured logic to support known or conventional operating features that are not described in detail herein. Although the server 104 is illustrated and described to be implemented within a single communication device, it is contemplated that the one or more components of the server 104 are alternatively implemented in a distributed computing environment. It will further be appreciated by those of ordinary skill in the art that one or more components of the server 104 alternatively functions within a remote server, cloud computing device, or any other remote computing mechanism now known or developed in the future. In some embodiments, one or more functions of the server 104 are distributed between two or more devices.

As illustrated in FIG. 3, the server 104 includes a plurality of electrical and electronic components, providing power, operational control, communication, and the like within the server 104. For example, the server 104 includes, among other components, a server transceiver 132, a server display 134, a server user interface 136, a server processor 138, and a server memory 140. It should be appreciated by those of ordinary skill in the art that FIG. 3 depicts the server 104 in a simplified manner and a practical embodiment includes additional components and suitably configured logic to support known or conventional operating features that are not described in detail herein. It will further be appreciated by those of ordinary skill in the art that the server 104 is a personal computer, desktop computer, tablet, smartphone, or any other computing device now known or developed in the future.

The components of the server 104, including the server transceiver 132, the server display 134, the server user interface 136, the server processor 138, and the server memory 140 communicates with one another via a server local interface 142. The server local interface 142 includes, namely, but not limited to, one or more buses or other wired or wireless connections, as is known in the art or developed in the future. The server local interface 142 have additional elements, but not limited to, such as controllers, buffers (caches), drivers, repeaters, and receivers, among many others, to enable communications. Further, the server local interface 142 includes address, control, and/or data connections to enable appropriate communications among the aforementioned components.

As illustrated, the server 104 includes the server transceiver 132 to receive the one or more inputs from and transmit the one or more outputs to the user device 106 (illustrated in FIG. 1). For example, the server transceiver 132 is configured to receive the query indicating the occurrence of the variance event from and/or transmit the response associated with the query to the user device 106. In some embodiments, the server transceiver 132 is configured to receive the feedback associated with the accuracy of the response from the user device 106. The server transceiver 132 includes a transmitter circuitry and a receiver circuitry to enable the server 104 to communicate with the user devices 106. In this regard, the transmitter circuitry includes appropriate circuitry to transmit the one or more outputs to the user devices 106 and the receiver circuitry includes appropriate circuitry to receive the one or more inputs from the user devices 106. It will be appreciated by those of ordinary skill in the art that the server 104 includes a single server transceiver as illustrated, or alternatively separate transmitting and receiving components, for example but not limited to, a transmitter, a transmitting antenna, a receiver, and a receiving antenna.

The server user interface 136 is configured to receive data from and/or provide an output to a user (for example, a programmer). The data is provided via a touch screen display (such as, the server display 134), a camera, a touch pad, a keyboard, a microphone, a recorder, a mouse, or any other user input mechanism now known or developed in the future. The output is provided via a display device, such as the server display 134, a speaker, a haptic output, or any other output mechanism now known or developed in the future. The server user interface 136 further includes a serial port, a parallel port, an infrared (IR) interface, a universal serial bus (USB) interface and/or any other interface herein known or developed in the future. The server display 134 includes a display screen, or a computer monitor now known or in the future developed.

The server memory 140 is a non-transitory memory configured to store a set of instructions that are executable by the server processor 138 to perform the predetermined operations. For example, the server memory 140 includes any of the volatile memory elements (for example, random access memory (RAM)), non-volatile memory elements (for example read only memory (ROM)), and combinations thereof. Moreover, the server memory 140 incorporates electronic, magnetic, optical, and/or other types of storage media. In some embodiments, the server memory 140 has a distributed architecture, where various components are situated remotely from one another, but are accessed by the server processor 138. The software in the server memory 140 includes one or more software programs, each of which includes an ordered listing of executable instructions for implementing logical functions.

In accordance with various embodiments, the server memory 140 includes a fine-tuned large language model (LLM) 144 that is obtained by fine-tuning a pre-trained LLM on a plurality of historical datasets. The historical dataset includes one or more of: a plurality of surgical profiles identifying one or more steps of a plurality of medical procedures, a facility knowledge dataset associated with a plurality of medical facilities, a surgeon knowledge dataset associated with a plurality of medical practitioners, and insights. For example, each surgical profile of the plurality of surgical profile is associated with a medical practitioner identifier identifying a medical practitioner, a medical procedure identifier identifying a medical procedure, a facility identifier identifying a medical facility, and a medical room identifier identifying a medical room in a corresponding medical facility. Further, the facility knowledge dataset includes data associated with a layout and a setup of one or more rooms in each medical facility of the plurality of medical facilities. In an exemplary embodiment, the surgeon knowledge dataset is classified based on genealogy of the plurality of medical practitioners. The surgeon knowledge dataset includes common medical practices followed by different sets of medical practitioners classified based on their genealogy. Further, the insights correspond to insights from a plurality of historical medical procedures performed by each medical practitioner of the plurality of medical practitioners. In some embodiments (not illustrated), the server memory 140 also stores the pre-trained LLM and/or the historical datasets described above. In some alternate embodiments, the pre-trained LLM and/or the historical datasets are stored on an external device (not illustrated) and accessed by the server processor 138, via the server transceiver 132. Furthermore, in some embodiments, the server memory 140 includes one or more natural language processing (NLP) models 146 to process the queries received from the user devices 106.

The server processor 138 is configured to execute the instructions stored in the server memory 140 to perform the predetermined operations. The server processor 138 includes one or more microprocessors, microcontrollers, DSPs (digital signal processors), state machines, logic circuitry, or any other device or devices that process information or signals based on operational or programming instructions. The server processor 138 is implemented using one or more controller technologies, such as Application Specific Integrated Circuit (ASIC), Reduced Instruction Set Computing (RISC) technology, Complex Instruction Set Computing (CISC) technology, or any other technology now known or in the future developed. The server processor 138 is configured to cooperate with other components of the server 104 to perform operations as described in detail with reference to FIG. 5. In some embodiments, when the query includes the data associated with the occurrence of the variance event in the medical room of the medical facility captured by the sensors 102, the server processor 138 is configured to process the query and identify the variance event (for example, any deviation, discrepancy, or unexpected occurrence from the standard, planned, or expected course of medical care, set-up, treatment, or procedure) based on a comparison of the captured data with the standard, planned, or expected course of medical care, set-up, treatment, or procedure.

In accordance with various embodiments, the server processor 138 includes an artificial intelligence (AI) engine 150. In accordance with various embodiments, the AI engine 150 receives the query from the user device 106. The AI engine 150 includes a LLM module 152. The LLM module 152 is any system configured to learn and adapt itself to do better in changing environments. The LLM module 152 employs any one or combination of the following computational techniques: neural network, constraint program, fuzzy logic, classification, conventional artificial intelligence, symbolic manipulation, fuzzy set theory, evolutionary computation, cybernetics, data mining, approximate reasoning, derivative-free optimization, decision trees, and/or soft computing. The LLM module 152 implements an iterative learning process. The learning is based on a wide variety of learning rules or training algorithms. The learning rules include one or more of back-propagation, pattern-by-pattern learning, supervised learning, and/or interpolation. The LLM module 152 is configured to implement one or more AI algorithms to fine-tune the pre-trained LLM based on a collective knowledge derived from the plurality of surgical profiles, the facility knowledge dataset, the surgeon knowledge dataset, and the insights to obtain the fine-tuned LLM 144 for generation of the response associated with the query. The pre-trained LLM is capable of performing a vast variety of tasks associated with the healthcare, and fine-tuning the pre-trained LLM is the process of adapting the pre-trained LLM to perform better on a specific task (for example, managing the variance events in the healthcare) by training it on a smaller, specialized dataset associated with the healthcare. In accordance with some embodiments of the invention, the AI algorithm utilizes any machine learning methodology, now known or in the future developed, for classification. For example, the machine learning methodology utilized includes one or a combination of: Linear Classifiers (Logistic Regression, Naive Bayes Classifier); Nearest Neighbor; Support Vector Machines; Decision Trees; Boosted Trees; Random Forest; and/or Neural Networks. The LLM module 152 continually fine-tunes the fine-tuned LLM 144 based on additional dataset and the feedback obtained from the user device 106 associated with the accuracy of the response.

In some embodiments, the AI engine 150 includes an NLP module 154. The NLP module 154 is trained to apply a natural language processing to an unstructured human language data to convert the unstructured human language data into a machine-readable structured data and vice-versa. In accordance with various embodiments, the unstructured human language data is obtained from the sensors 102 via the user device 106 or directly from the user device 106. In accordance with various embodiments, the NLP module 154 is configured to apply the natural language processing to the query including a natural language input to convert the natural language input into a format understandable by the fine-tuned LLM 144. For example, the natural language input corresponds to a text string or a voice input representing the query indicative of the occurrence of the variance event. The NLP module 154 is any system configured to learn and adapt itself to do better in changing environments. The NLP module 154 is configured to implement one or more NLP algorithms to train the NLP models 146 to convert the natural language input into the format understandable by the fine-tuned LLM 144.

In some embodiments, the AI engine 150 includes a computer vision module 156. The computer vision module 156 is configured to analyze, recognize, and extract information from any visual information, for example, an image, a video, and/or real-world environment, utilizing one or more computer vision techniques. As discussed above, the visual information is captured by the one or more sensors 102 or the user device 106. In accordance with various embodiments, the computer vision module 156 is configured to obtain the visual information associated with the query and process the visual information to obtain additional data associated with the query. For example, the computer vision module 156 is configured to detect and recognize objects, faces, and patterns in the received visual information. The computer vision module 156 is configured to implement the one or more computer vision techniques known in the art or developed in the future to analyze, recognize, and extract information from the visual information.

The server processor 138 further includes a command module 158 and a response module 160 to obtain contextual information associated with a context of the query received from the user device 106. For example, the command module 158 is configured to obtain the visual information, via the image capturing device 102-1, to obtain a visual context associated with the query. In some embodiments, the image capturing device corresponds to the camera of the user device 106. In some embodiments, the command module 158 is also configured to obtain data associated with the user device GUI 122 (interchangeably referred to as application windows context) accessed via the user device 106. The data indicates one or more of an open window in the user device GUI 122 and one or more buttons (for example, the microphone button, the camera button, or the file upload button) selected on the user device GUI 122. Further, the response module 160 is configured to obtain a current contextual information associated with the query based on the conversation with the user device 106. For example, the current contextual information corresponds to an emotional context associated with providing the query.

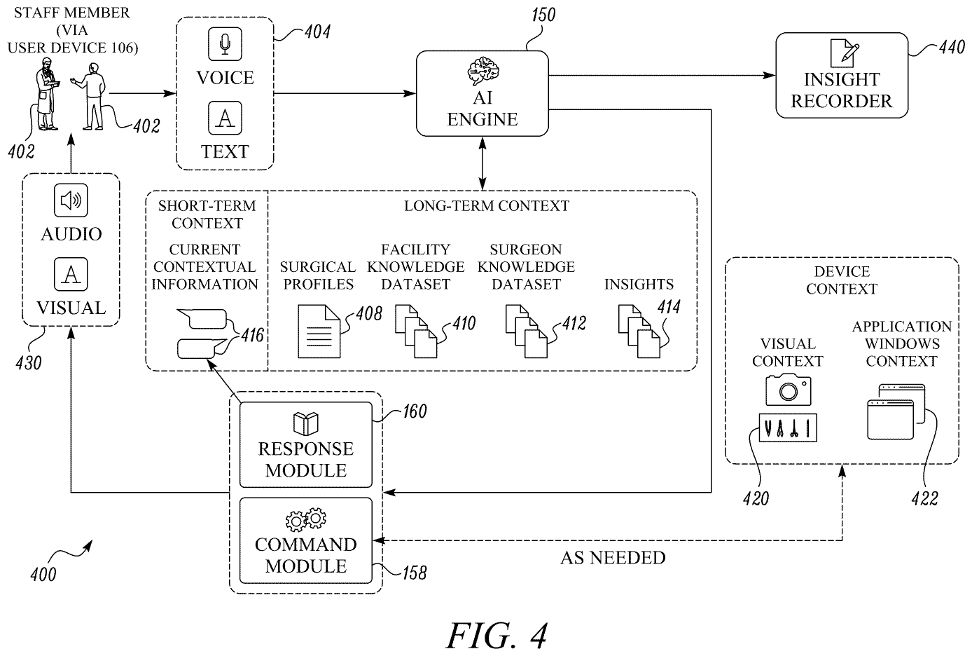

Referring to FIG. 5, an exemplary method 500 for managing variance events in healthcare is illustrated and described. The method 500 is also discussed in conjunction with FIG. 4. The method 500 begins, at 502, with the user device 106 receiving the data indicating the occurrence of the variance event in the medical room of the medical facility from the one or more sensors 102. As discussed above, the data corresponds to the visual data, the audio data or any data capturing any deviation, discrepancy, or unexpected occurrence from the standard, planned, or expected course of medical care, treatment, or procedure. At 504, the user device 106 formulates the query indicating the occurrence of the variance event in the medical room of the medical facility based on the received data. In an embodiment, the query includes the data associated with the occurrence of the variance event in the medical room of the medical facility captured by the sensors 102. In some embodiments, the user device 106 formulates the query by comparing the captured data with the standard, planned, or expected course of medical care, set-up, treatment, or procedure and determining any deviation, discrepancy, or unexpected occurrence from the standard, planned, or expected course of medical care, set-up, treatment, or procedure based on the comparison. The user device 106 then formulates the query based on the determined deviation, discrepancy, or unexpected occurrence. In some embodiments, the user device processor 116 is configured to receive the query and/or the supplement data associated with the query from its user.

In accordance with various embodiments, the query corresponds to a request to provide with the one or more actions to be performed to manage the variance event. For example, the query describes that the vital signs of a patient have fallen outside the expected range during the medical procedure and requests the one or more actions to be performed to manage the variance event. In some embodiments, the query also indicates one or more of a medical practitioner identifier identifying a medical practitioner associated with the medical procedure or the patient, a medical procedure identifier identifying the medical procedure performed or to be performed on the patient, a medical facility identifier identifying the medical facility associated with the variance event, and a medical room identifier identifying the medical room associated with the variance event. The query can include the natural language input in a form of the text string or the voice input. For example, FIG. 4 illustrates a process map 400 with a staff member 402 providing the query 404, via its user device 106, indicating the occurrence of the variance event.

At 506, the server processor 138 of the server 104 accesses a LLM (interchangeably referred to as pre-trained LLM) associated with the healthcare. In some embodiments, the pre-trained LLM is stored in the server memory 140. In some other embodiments, the pre-trained LLM is stored on an external device (not illustrated) and accessed by the server processor 138, via the server transceiver 132. At 508, the LLM module 152 of the server processor 138 fine-tunes the pre-trained LLM based on the plurality of historical datasets to provide a long-term context to the pre-trained LLM. The historical dataset includes one or more of: the plurality of surgical profiles identifying one or more steps of the plurality of medical procedures, the facility knowledge dataset associated with the plurality of medical facilities, the surgeon knowledge dataset associated with the plurality of medical practitioners, and the insights.

In accordance with various embodiments, each surgical profile of the plurality of surgical profile is associated with a medical practitioner identifier identifying a medical practitioner, a medical procedure identifier identifying a medical procedure, a facility identifier identifying a medical facility, and a medical room identifier identifying a medical room in a corresponding medical facility. For example, a first surgical profile identifies one or more steps of a heart surgery associated with a medical practitioner ‘A’ in a medical room ‘X’ of a facility ‘Y’. Similarly, a second a profile identifies one or more steps of a spinal surgery associated with a medical practitioner ‘B’ in a medical room ‘W’ of a facility ‘Z’. Further, the facility knowledge dataset includes data associated with a layout and a setup of one or more rooms in each medical facility of the plurality of medical facilities. For example, the facility knowledge dataset indicates that the medical room ‘X’ of the medical facility ‘Z’ includes a ventilator. In an exemplary embodiment, the surgeon knowledge dataset is classified based on genealogy of the plurality of medical practitioners. The surgeon knowledge dataset indicates common medical practices followed by different sets of medical practitioners classified based on their background, for example but not limited to, the medical college and training institutions attended, geographical location of the education and current practice, including the medical facility where they are presently practicing. For example, the surgeon knowledge dataset indicates that the medical practitioner ‘A’ completed his medical education from the medical college ‘YY’ and that the medical practitioners from the medical college ‘YY’ are well versed with the usage of advanced robotics. Further, the insights correspond to insights from the plurality of historical medical procedures performed by each medical practitioner of the plurality of medical practitioners. For example, the insights indicate that the medical practitioner ‘A’ deploys the stent 10 minutes into the heart surgery. For example, FIG. 4 illustrates the AI engine 150 of the server processor 138 utilizing the surgical profiles 408, the facility knowledge dataset 410, the surgeon knowledge dataset 412, and the insights 414 to provide the long-term context to the pre-trained LLM.

In some embodiments, the LLM module 152 of the server processor 138 further fine-tunes the fine-tuned LLM 144 based on the current contextual information to provide the short-term context to the fine-tuned LLM 144. The current contextual information corresponds to the emotional context associated with providing the query. For example, the server processor 138 or the LLM module 152 of the server processor 138 obtains the current contextual information associated with the query via the response module 160 and further fine-tunes the fine-tuned LLM 144 based on the current contextual information. For example, FIG. 4 illustrates the AI engine 150 of the server processor 138 utilizing the current contextual information 416 from the response module 160 to provide the short-term context to the pre-trained LLM.

At 510, the server processor 138 of the server 104 provides the query to the fine-tuned LLM 144. To this end, the server processor 138 is configured to provide the query to the LLM module 152 that executes the fine-tuned LLM 144. In some embodiments, the server processor 138 pre-processes the query in the format understandable by the fine-tuned LLM 144 prior to providing the query to the fine-tuned LLM 144. For example, the NLP module 154 of the server processor 138 pre-processes the query by applying the natural language processing to the natural language input associated with the query to convert the natural language input into the format understandable by the fine-tuned LLM 144. It would be appreciated by a person skilled in the art that the conversion of the natural language input into the machine-readable data is well-known in the art and hence is not described here for the sake of brevity.

In some embodiments, when the query includes the data associated with the occurrence of the variance event in the medical room of the medical facility captured by the sensors 102, the server processor 138 processes the query and identifies the variance event (for example, any deviation, discrepancy, or unexpected occurrence from the standard, planned, or expected course of medical care, set-up, treatment, or procedure) based on the comparison of the captured data with the standard, planned, or expected course of medical care, set-up, treatment, or procedure.

In some embodiments, the server processor 138, upon receiving the query, processes the query to determine whether a visual information associated with the query is required. To this end, the server processor 138 processes the query to determine whether any additional information that must be gathered visually is required to provide the response to the query. For example, when the query corresponds to ‘which stent from the tray is to be deployed?’, the server processor 138 determines that additional information associated with the visual of the tray is needed to provide the response to the query. When the visual context is required, the command module 158 of the server processor 138 obtains, via an image capturing device, the visual information associated with the query. In accordance with various embodiments, the image capturing device corresponds to the camera of the user device 106 or any image capturing device installed in the medical room. The server processor 138, via the computer vision module 156, processes the visual information utilizing the one or more computer vision techniques to obtain additional data associated with the query. The server processor 138 then supplements the query with the additional data prior to providing the query to the fine-tuned LLM 144. In accordance with various embodiments, the additional data is in the format understandable by the fine-tuned LLM 144.

In some embodiments, the server processor 138 of the server processor 138 also obtains the data associated with the user device GUI 122 accessed via the user device 106 via the command module 158. The server processor 138 then provides the obtained data to the fine-tuned LLM 144 to provide the contextual information associated with the context of the query to the fine-tuned LLM 144. For example, FIG. 4 illustrates the command module 158 obtaining the visual context 420 and the application windows context 422.

At 512, the LLM module 152 of the server processor 138 generates the response associated with the query from the fine-tuned LLM 144. The response is generated based on a collective knowledge derived by the fine-tuned LLM 144 from the plurality of surgical profiles, the facility knowledge dataset, the surgeon knowledge dataset, and the insights. The response indicates one or more actions to be performed to manage the variance event. In accordance with various embodiments, the response corresponds to instructions to modify a standard process to be followed for a patient in the medical room. For example, the one or more actions indicate administering additional medications and waiting for a predefined time period to stabilize the vital signs of the patient and then proceeding with the next step of the medical procedure (instead of directly proceeding with the next step of the medical procedure, which is a standard procedure). The actions also identify the medication, an amount of the medication to be administered, the predefined time period, the normal vital signs of the patient, and the next step of the medical procedure.

For example, the LLM module 152 generates the response by obtaining a first medical practitioner identifier identifying a medical practitioner, a first medical procedure identifier identifying a medical procedure, a first medical facility identifier identifying a medical facility, a first medical room identifier identifying a medical room associated with the variance event. For example, the LLM module 152 identifies the first medical practitioner identifier as the medical practitioner ‘A’, the first medical procedure identifier as the heart surgery, the first medical facility identifier as the medical facility ‘Y’, and the first medical room identifier as the medical room ‘W’. In some embodiments, the server processor 138 requests the first medical practitioner identifier, the first medical procedure identifier, the first medical facility identifier, and the first medical room identifier from the user device 106. In some alternate embodiments, the server processor 138 obtains the first medical practitioner identifier, the first medical procedure identifier, the first medical facility identifier, and the first medical room identifier directly from one or more external devices, for example, augmented reality devices in the medical room.

The LLM module 152 then identifies and analyzes one or more of: a surgical profile of the plurality of surgical profiles, a first subset of the facility knowledge dataset, a first subset of the surgeon knowledge dataset, and the insights associated with one or more of the first medical identifier, the first medical procedure identifier, the first medical facility identifier, and the first medical room identifier. In the example illustrated above, the LLM module 152 identifies and analyzes one or more of the surgical profile, the first subset of the facility knowledge dataset, the first subset of the surgeon knowledge dataset, and the insights associated with one or more of the medical practitioner ‘A’, the heart surgery, the medical facility ‘Y’, and the medical room ‘W’. The LLM module 152 further generates the response associated with the query based on the surgical profile, the first subset of the facility knowledge dataset, the first subset of the surgeon knowledge dataset, and the insights. In accordance with various embodiments, the generated response is associated with the first medical identifier, the first medical procedure identifier, the first medical facility identifier, and the first medical room identifier and indicates one or more actions specific to the first medical identifier, the first medical procedure identifier, the first medical facility identifier, and the first medical room identifier.

The LLM module 152 modifies the response based on the current contextual information. In accordance with various embodiments, modifying the response includes at least one of: modifying a mode of presenting the response on the user device 106, modifying a tone of presenting the response on the user device 106, modifying a pace of presenting the response on the user device 106, and modifying an extent of details to be provided in the response on the user device 106. For example, when the current contextual information indicates that the user is nervous, the LLM module 152 modifies the mode and/or tone of presenting the response to create a supportive environment for the user.

At 514, the server processor 138 controls the one or more medical devices in the medical room to manage the variance event based on the response. In accordance with various embodiments (not illustrated), the medical devices are operatively and communicatively coupled to the server 104 to enable the server 104 to control one or more operations of the medical devices based on the one or more actions. For example, the server processor 138 is operatively coupled to the intravenous device to control the intravenous device to administer the additional medications to the patient based on the one or more actions. It would be appreciated by a person skilled in the art that the medical device can include any medical device used in the healthcare sector, for example but not limited to, anesthesia systems, electrocautery systems, C-arm systems, enabling technology systems or workstations (for example, but not limited to, a microscope, an electrocardiogram device, a robotic surgical system, a navigation system, a networked robotics, an illumination system, or any other enabling technology systems now known or in the future developed, and accompanying monitors or displays), a blood pressure monitoring machine, a heart rate monitoring machine, and various other medical devices (not illustrated) now known or in the future developed.

The server processor 138 presents the response on the user device 106 for its corresponding user. As discussed above, the response indicated the one or more actions to be performed to manage the variance events. In some embodiments, the server processor 138 presents the response on the user device 106 to obtain approval from the user prior to controlling the one or more medical devices to manage the variance event. In some embodiments, the server processor 138 augments the response from the fine-tuned LLM 144 by converting the response into at least one of a visual output or an audio output. For example, when the user device 106 corresponds to the augmented reality device 106-4, the server processor 138 provides virtual representation of the one or more actions to be performed to manage the variance events on the augmented reality device 106-4. FIG. 4 illustrates transmitting the response 430 (in the visual output and/or the audio output format) to the user device 106 of the staff member 402. The user device 106, upon receiving the response, presents the augmented response on the user device display 114. In some embodiments, the server processor 138 obtains feedback associated with an accuracy of the response via the user device 106 and fine-tunes the fine-tuned LLM 144 based on the obtained feedback. In some embodiments, the response and the feedback are recorded by the server processor 138 in the server memory 140 as an insight recorder for further fine-tuning of the fine-tuned LLM 144. For example, FIG. 4 illustrates the insight recorder 440 recording the outputs from the AI engine 150.

The system and method of the present disclosure enable medical practitioners and/or staff to handle the variance events confidently, ensuring continuity of care even when experienced medical practitioners are unavailable. By utilizing a collective knowledge derived from the plurality of surgical profiles, the facility knowledge dataset, the surgeon knowledge dataset, and the insights, the generated response ensures effective management of the variance events. Moreover, the generated response is also modified based on the emotional context associated with providing the query to create a supportive environment for the user.

In the hereinbefore specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. The benefits, advantages, solutions to problems, and any element(s) that can cause any benefit, advantage, or solution to occur or become more pronounced are not to be construed as a critical, required, or essential feature or elements of any or all the claims. The invention is defined solely by the appended claims including any amendments made during the pendency of this application and all equivalents of those claims as issued.

Moreover, in this document, relational terms such as first and second, top and bottom, and the like are used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but includes other elements not expressly listed or inherent to such process, method, article, or apparatus. An element preceded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way but also be configured in ways that are not listed.

It will be appreciated that some embodiments are comprised of one or more generic or specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the method and/or apparatus described herein. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used.

Moreover, an embodiment can be implemented as a computer-readable storage medium having computer readable code stored thereon for programming a computer (example, comprising a processor) to perform a method as described and claimed herein. Examples of such computer-readable storage mediums include, but are not limited to, a hard disk, a CD-ROM, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation.

1. A system for managing variance events in healthcare, the system comprising:
a user device configured to:
receive data indicating an occurrence of a variance event in a medical room of a medical facility; and
formulate a query indicating the occurrence of the variance event in the medical room of the medical facility based on the received data; and
a server communicatively coupled to the user device, the server configured to:
access a large language model (LLM) associated with the healthcare;
fine-tune the LLM based on a plurality of historical datasets to provide a long-term context to the LLM, wherein the historical dataset includes
a surgeon knowledge dataset associated with a plurality of medical practitioners, wherein the surgeon knowledge dataset is classified based on genealogy %of the plurality of medical practitioners, the genealogy including a background of each medical practitioner of the plurality of medical practitioners, the background including: a medical college attended, a training institution attended, and a geographical location of education, wherein the surgeon knowledge dataset includes common medical practices followed by different sets of medical practitioners classified based on their genealogy;
provide the query to the fine-tuned LLM;
generate a response associated with the query from the LLM, wherein the response indicates one or more actions to be performed to manage the variance event; and
automatically transmit control signals to one or more medical devices in the medical room based on the generated response; and
the one or more medical devices in the medical room communicatively coupled to the server, the one or more medical devices are configured to:
receive the control signals from the server; and
automatically adjust operational parameters of the one or more medical devices in real-time based on the received control signals to manage the variance event.

2. The system of claim 1, wherein the response corresponds to instructions to modify a standard process to be followed for a patient in the medical room.

3. The system of claim 1, wherein the server is configured to present, via the user device, the response to a user of the user device.

4. The system of claim 3, wherein the server is further configured to:
fine-tune the LLM based on a current contextual information to provide a short-term context to the LLM, wherein the current contextual information corresponds to an emotional context associated with providing the query; and
modify the response based on the current contextual information.

5. The system of claim 4, wherein modifying the response includes at least one of: modifying a mode of presenting the response on the user device, modifying a tone of presenting the response, modifying a pace of presenting the response, and modifying an extent of details to be provided in the response.

6.-7. (canceled)

8. The system of claim 1, wherein the server is further configured to:
process the query to determine whether a visual information associated with the query is required;
obtain, via an image capturing device, the visual information associated with the query when the visual information is required;
process the visual information utilizing one or more computer vision techniques to obtain additional data associated with the query; and
supplement the query with the additional data prior to providing the query to the fine-tuned LLM.

9. The system of claim 1, wherein the server is configured to pre-process the query in a format understandable by the fine-tuned LLM prior to providing the query to the fine-tuned LLM.

10. The system of claim 9, wherein the query includes a natural language input, and wherein the server is configured to pre-process the query by:
applying a natural language processing to the natural language input to convert the natural language input into a format understandable by the fine-tuned LLM.

11. The system of claim 1, wherein the server is configured to:
augment the response from the fine-tuned LLM by converting the response into at least one of a visual output or an audio output; and
wherein the user device is configured to:
present the augmented response received from the server.

12. The system of claim 1, wherein the server is configured to:
obtain feedback associated with an accuracy of the response via the user device; and
fine-tune the LLM based on the obtained feedback.

13. A method for managing variance events in healthcare, the method comprising:
receiving, by a user device, data indicating an occurrence of a variance event in a medical room of a medical facility;
formulating, by the user device, a query indicating the occurrence of the variance event in the medical room of the medical facility based on the received data;
accessing, by a server, a large language model (LLM) associated with the healthcare;
fine-tuning, by the server, the LLM based on a plurality of historical datasets to provide a long-term context to the LLM, wherein the historical dataset includes
a surgeon knowledge dataset associated with a plurality of medical practitioners, wherein the surgeon knowledge dataset is classified based on genealogy of the plurality of medical practitioners, the genealogy including a background of each medical practitioner of the plurality of medical practitioners, the background including: a medical college attended, a training institution attended, and a geographical location of education, wherein the surgeon knowledge dataset includes common medical practices followed by different sets of medical practitioners classified based on their genealogy;
providing, by the server, the query to the fine-tuned LLM;
generating, by the server, a response associated with the query from the LLM, wherein the response indicates one or more actions to be performed to manage the variance event;
automatically transmitting, by the server, control signals to one or more medical devices in the medical room based on the generated response;
receiving, by the one or more medical devices in the medical room communicatively coupled to the server, the control signals from the server; and
automatically adjusting. by the one or more medical devices, operational parameters of the one or more medical devices in real-time based on the received control signals to manage the variance event.

14. The method of claim 13, wherein the response corresponds to instructions to modify a standard process to be followed for a patient in the medical room.

15. The method of claim 13, further including:
presenting, by the server, the response to a user of the user device via the user device.

16. The method of claim 15, further including:
fine-tuning, by the server, the LLM based on a current contextual information to provide a short-term context to the LLM, wherein the current contextual information corresponds to an emotional context associated with providing the query; and
modifying, by the server, the response based on the current contextual information,
wherein modifying the response includes at least one of: modifying a mode of presenting the response on the user device, modifying a tone of presenting the response, modifying a pace of presenting the response, and modifying an extent of details to be provided in the response.

17.-18. (canceled)

19. The method of claim 13, further including:
processing, by the server, the query to determine whether a visual information associated with the query is required;
obtain, by the server, the visual information associated with the query via an image capturing device when the visual information is required;
processing, by the server, the visual information utilizing one or more computer vision techniques to obtain additional data associated with the query; and
supplementing, by the server, the query with the additional data prior to providing the query to the fine-tuned LLM.

20. The method of claim 13, further including:
pre-processing, by the server, the query in a format understandable by the fine-tuned LLM prior to providing the query to the fine-tuned LLM.

21. The system of claim 1, wherein the one or more actions include administering additional medications to a patient; and wherein the one or more medical devices include an intravenous device in the medical room, wherein the intravenous device is operatively and communicatively coupled to the server, and wherein controlling the intravenous device includes automatically adjusting the intravenous device to administer the additional medications to the patient based on the one or more actions indicated in the response.

22. The method of claim 13, wherein the one or more actions include administering additional medications to a patient; and wherein the one or more medical devices include an intravenous device in the medical room, and wherein controlling the intravenous device includes automatically adjusting the intravenous device to administer the additional medications to the patient based on the one or more actions indicated in the response.

23. The system of claim 1, wherein the one or more actions indicated in the response include administering additional medications and waiting for a predefined time period to stabilize vital signs of a patient prior to proceeding with a next step of a medical procedure, and wherein the response identifies the additional medications, an amount of the additional medications to be administered, the predefined time period, normal vital signs of the patient, and the next step of the medical procedure.

24. The method of claim 13, wherein the one or more actions indicated in the response include administering additional medications and waiting for a predefined time period to stabilize vital signs of a patient prior to proceeding with a next step of a medical procedure, and wherein the response identifies the additional medications, an amount of the additional medications to be administered, the predefined time period, normal vital signs of the patient, and the next step of the medical procedure.

25. The system of claim 1, wherein the historical dataset further includes:
a plurality of surgical profiles identifying one or more steps of a plurality of medical procedures, each surgical profile of the plurality of surgical profile is associated with a medical practitioner identifier identifying a medical practitioner, a medical procedure identifier identifying a medical procedure, a facility identifier identifying a medical facility, and a medical room identifier identifying a medical room in a corresponding medical facility,
a facility knowledge dataset associated with a plurality of medical facilities, wherein the facility knowledge dataset includes data associated with a layout and a setup of one or more rooms in each medical facility of the plurality of medical facilities, and
insights from a plurality of historical medical procedures performed by each medical practitioner of the plurality of medical practitioners.

26. The method of claim 13, wherein the historical dataset further includes:
a plurality of surgical profiles identifying one or more steps of a plurality of medical procedures, each surgical profile of the plurality of surgical profile is associated with a medical practitioner identifier identifying a medical practitioner, a medical procedure identifier identifying a medical procedure, a facility identifier identifying a medical facility, and a medical room identifier identifying a medical room in a corresponding medical facility,
a facility knowledge dataset associated with a plurality of medical facilities, wherein the facility knowledge dataset includes data associated with a layout and a setup of one or more rooms in each medical facility of the plurality of medical facilities, and
insights from a plurality of historical medical procedures performed by each medical practitioner of the plurality of medical practitioners.

27. The system of claim 25, wherein the response is generated based on a collective knowledge derived by the fine-tuned LLM from the plurality of surgical profiles, the facility knowledge dataset, the surgeon knowledge dataset, and the insights.

28. The system of claim 25, wherein the server is configured to generate the response by:
obtaining a first medical practitioner identifier, a first medical procedure identifier, a first medical facility identifier, a first medical room identifier associated with the variance event;
identifying and analyzing one or more of: a surgical profile of the plurality of surgical profiles, a first subset of the facility knowledge dataset, a first subset of the surgeon knowledge dataset, and the insights associated with the first medical practitioner identifier, the first medical procedure identifier, the first medical facility identifier, and the first medical room identifier; and
generating the response associated with the query based on the surgical profile, the first subset of the facility knowledge dataset, the first subset of the surgeon knowledge dataset, and the insights.

29. The method of claim 26, wherein the response is generated based on a collective knowledge derived by the fine-tuned LLM from the plurality of surgical profiles, the facility knowledge dataset, the surgeon knowledge dataset, and the insights.

30. The method of claim 26, wherein generating the response includes:
obtaining a first medical practitioner identifier, a first medical procedure identifier, a first medical facility identifier, a first medical room identifier associated with the variance event;
identifying and analyzing one or more of: a surgical profile of the plurality of surgical profiles, a first subset of the facility knowledge dataset, a first subset of the surgeon knowledge dataset, and the insights associated with the first medical practitioner identifier, the first medical procedure identifier, the first medical facility identifier, and the first medical room identifier; and
generating the response associated with the query based on the surgical profile, the first subset of the facility knowledge dataset, the first subset of the surgeon knowledge dataset, and the insights.
